SEQUENCE LISTING (6 sequences)

SEQ ID NO: 1 - protein, 8 aa, Artificial Sequence
  Cyclic Protein 11
CGWIYWNV

SEQ ID NO: 2 - protein, 14 aa, Artificial Sequence
  Linear Tat peptide
CGRKKRRQRRRPPQ

SEQ ID NO: 3 - DNA, 26 bp, Artificial Sequence
  forward primer for p6 HEV
atgcgaattccgcgccgttgtaacct

SEQ ID NO: 4 - DNA, 16 bp, Artificial Sequence
  reverse primer for p6 HEV
accgggacagcgtgga

SEQ ID NO: 5 - DNA, 20 bp, Artificial Sequence
  forward primer for GAPDH RNA
gagtcaacggatttggtcgt

SEQ ID NO: 6 - DNA, 20 bp, Artificial Sequence
  reverse primer for GAPDH RNA
ttgattttggagggatctcg

(19) United States
(12) Patent Application Publication
(10) Pub. No.: US 2020/0115417 A1
(43) Pub. Date: Apr. 16, 2020

(54) A CYCLIC PEPTIDE AND PHARMACEUTICAL COMPOSITION COMPRISING THE SAME FOR INHIBITING PROLIFERATION OF HEV

(71) Applicant: TRANSLATIONAL HEALTH SCIENCE AND TECHNOLOGY INSTITUTE, Faridabad (IN)

(72) Inventors: Surjit MILAN, Faridabad (IN); Saumya ANANG, Faridabad (IN)

(73) Assignee: TRANSLATIONAL HEALTH SCIENCE AND TECHNOLOGY INSTITUTE, Faridabad (IN)

(21) Appl. No.: 16/627,516

(22) PCT Filed: Jul. 23, 2018
(86) PCT No.: PCT/IN2018/050475

§ 371 Date: Dec. 30, 2019

(30) Foreign Application Priority Data
Feb. 9, 2018 (IN) 201811004996

(51) Int. Cl. C07K 7/64; A61K 38/00

(52) U.S. Cl. CPC C07K 7/64; A61K 38/00

(57) ABSTRACT

An objective of the present invention is to provide a means for inhibiting progression of HEV infection in a host by use of a cyclic peptide including the amino acid sequence of SEQ ID No: 1 which inhibits interaction between HEV ORF3 domain and host TSG101 which is crucial for HEV proliferation in a host or an expression vector coding for the cyclic peptide or a pharmaceutical composition comprising the cyclic peptide with pharmaceutically acceptable carriers.

### TECHNICAL FIELD

The present invention relates to a cyclic peptide which is useful in inhibiting interaction between HEV ORF3 domain and host TSG101, a pharmaceutical composition comprising the same and a method of utilizing the same for inhibiting proliferation of HEV in a host.

### BACKGROUND ART

Hepatitis E virus (HEV) is a major cause of viral hepatitis. Though the infection is acute in normal individuals, it becomes chronic in immuno-compromised patients such as organ transplant recipients, HIV infected individuals and patients undergoing chemotherapy (1-6). The disease worsens in pregnancy with mortality rate as high as 20 to 25% (7-9). Recent reports indicate extra hepatic manifestations by HEV such as Guillan-Barre syndrome, neurological amyotrophy, arthritis, pancreatitis and glomerulonephritis (10-13). Outbreaks of HEV have been reported from different corners of the world. Several parts of eastern and central India and several parts of Africa have been affected with frequent HEV epidemic (14-18). Recent increase in organ transplantation and exposure to the disease due to growing trade and travel has further expanded the HEV infection, thereby intensifying the need of antiviral research against HEV.

Mammalian HEV is known to have 8 different genotypes (19). Genotype-1 and -2 (g-1, g-2) are restricted to humans, whereas genotype-3 and -4 (g-3, g-4) are zoonotic (1). Among these, g-1 is more prevalent in India and other Asian countries that constitute the HEV endemic areas (20). HEV g-1 and g-2 were responsible for about 20.1 million HEV infections, 3.4 million symptomatic cases, 70,000 fatalities and 30,000 still births in 2005 (20). G-3 is common in developed countries resulting in sporadic cases of HEV due to consumption of infected meat products (21, 22).

There is no specific drug against HEV (23). Supportive care is the option for majority of HEV cases. Broad spectrum antivirals such as pegylated-interferon alpha, ribavirin or a combination of both is the available therapeutic option in severe acute and chronic cases (24-31). The side effects of both ribavirin and interferon therapy render the treatment unsuitable for several categories of patients (29, 32-34). Recently, sofosbuvir, an anti-viral against HCV was reported to inhibit g-3 HEV replication in vitro, with an additive effect when combined with ribavirin (35). However, those data were not fully reproducible by Wang et al (36). Moreover, sofosbuvir treatment failed to clear HEV viremia in an immunosuppressed patient with chronic HCV and HEV without ribavirin (37). Hence, usage of sofosbuvir as an anti-HEV therapeutic needs further validation and there is an urgent need to identify new drug candidates for the treatment of Hepatitis E.

Inventors of the present invention were aware of the fact that protein-protein interactions (PPIs) in every living organism are essential for maintaining structural and functional integrity of that organism and these interactions also play crucial functions during HEV-host interactions (38). For harnessing important information hidden inside these PPIs during HEV-host interactions, inventors were causing perturbations in PPIs essential for the survival of HEV inside its host which led identification of a crucial interaction between HEV ORF3 and UEV domain of host TSG101 through its conserved P(T/S)AP motif (39). Subsequent studies demonstrated that the above mentioned interaction is essential for the release of g3-HEV (40, 41). With a view that the interaction between HEV ORF3 and host TSG101 is crucial for HEV budding and a candidate which can disrupt this interaction may be a potential candidate against HEV, inventors evaluated few cyclic peptides which were experimentally proven to inhibit HIV-1 gag-TSG101 interaction. Inventors found that out of several cyclic peptides screened, cyclic peptide 11 (CP11) inhibits the interaction between ORF3 and TSG101 proteins most efficiently and blocks the release of both g-1 and g-3 HEV by approximately 90% without showing any cytotoxicity. This observation is the object of this invention.

### DISCLOSURE OF THE INVENTION

### Problems to be Solved by the Invention

An objective of the present invention is to develop an agent which can inhibit proliferation of HEV in a host to stop Hepatitis E progression in the host. More specifically, an objective of the present invention is to identify peptides which can inhibit proliferation of HEV in a host to stop Hepatitis E progression in the host. The present invention enables inhibition of progression of HEV infection in a host through use of a peptide based bio-pharmaceutical.

### Means for Solving the Problems

Herein, the present inventors proved experimentally that a cyclic peptide of amino acid sequence Cys-Gly-Trp-Ile-Tyr-Trp-Asn-Val (SEQ ID NO:1) can inhibit interaction between HEV ORF3 domain and host TSG101.

As this interaction is essential for the proliferation of HEV in host cell, its inhibition by the cyclic peptide of the invention inhibits progression of Hepatitis E in a host.

More specifically, the present invention provides the followings:
(A) A cyclic peptide having the amino acid sequence Cys-Gly-Trp-Ile-Tyr-Trp-Asn-Val (SEQ ID NO:1) for inhibiting interaction between HEV ORF3 and host TSG101;
(B) An expression vector coding for the cyclic peptide of (A);
(C) A pharmaceutical composition for inhibiting HEV infection and proliferation comprising the cyclic peptide of (A) or the expression vector of (B) in an amount sufficient to inhibit HEV infection and proliferation; and a pharmaceutically-acceptable carrier.
(D) A method for treating Hepatitis E in a host, which comprises the step of administering the pharmaceutical composition of (C) to the subject in a dose sufficient to inhibit interaction between HEV ORF3 and host TSG101.
(E) Use of cyclic peptide of (A) or expression vector of (B), or pharmaceutical composition of (C) for inhibiting infection and proliferation of HEV in a host.
(F) Use of cyclic peptide of (A) or expression vector of (B) or pharmaceutical composition of (C) for inhibiting interaction between HEV ORF3 and host TSG101.

### BRIEF DESCRIPTION OF THE DRAWINGS

In the drawings:

FIG. 1 shows graphical representation of Western blot depicting inhibition of HEV ORF3 and host TSG101 interaction by CP11 (A) Left panel: Coomassie blue stained image of purified ORF3-His protein. Right panel: Western blot image of left panel sample, probed using anti-his antibody. (B) Western blot of TNT expressed proteins using anti-myc antibody. Mock lysate represents TNT of empty pGBKT7 vector. *: non-specific bands. (C) His-pull down assay. Upper panel: anti-myc western blot of ORF3-his bound proteins. Lower panel: anti-ORF3 western blot of aliquots of the sample represented in the upper panel. *: non-specific bands.

FIG. 2 shows graphical representation of inhibition of HEV virus egress in the mammalian cell culture system by CP11. (A) Viability measurement of p6 HEV expressing Huh7 cells, treated with CP11, as indicated. Values are mean±SEM of triplicate samples. (B) QRT-PCR measurement of the p6 HEV sense-strand RNA level in aliquots of sample shown in (A). HEV sense RNA values were normalized to that of GAPDH and represented as mean±SEM of triplicate samples. (C) QRT-PCR estimation of the p6 HEV genome copies in the culture medium of Huh7 cells used in (A) and (B). Values are means±SEM of triplicate samples. (D) ELISA of ORF2 VLPs using anti-ORF2 antibody. Values are mean±SEM of the corrected absorbance (A450-650) of triplicate samples. (E) ELISA p6 HEV secreted into the culture medium of Huh7 cells, treated with CP11, as indicated. Values are mean±SEM of the corrected absorbance (A450-650) of triplicate samples. (F) Measurement of secreted Gaussia luciferase in the culture medium of Huh7 cells expressing p6 HEV-Luc, treated with CP11, as indicated. Luc values were normalized to that of cell viability and represented as mean±SEM. G.QRT-PCR estimation of the g-1 HEV genome copies in the culture medium of ORF4-Huh7 cells, treated with CP11, as indicated. Values are mean±SEM of triplicate samples. H. Viability measurement of ORF4-Huh7 cells used in (G) Values are mean±SEM of triplicate samples.

### EXAMPLES

### Example 1

1.1. Materials

pSK HEV p6 and pSK p6 HEV-Luc (GenBank accession no. JQ679013.1) plasmids containing cDNA of p6 HEV and p6 HEV-Luc replicons were generously provided by Dr. S. Emerson and have been described previously (45, 46). pGBKT7 RdRp and pGBKT7 ORF4 plasmids have been described by us previously (47, 48). pGBKT7 TSG101 has been reported earlier (39). Anti-his (SC-57598), anti-myc (SC-789) antibodies were from Santa Cruz Biotechnology (Texas, USA). Anti-ORF2 antibody has been described earlier (48). Anti-ORF3 antibody was generously provided by Dr Shahid Jameel and has been reported previously (49). Huh7 human hepatoma cells were originally obtained from the laboratory of Prof. C. M. Rice.

1.2. Coupled In Vitro Transcription-Translation Assay

pGBKT7 RdRp, TSG101 and ORF4 plasmids, in which coding sequence for RdRp, TSG101 and ORF4 is cloned under the control of the T7 promoter along with a N-terminal myc epitope tag was used for in vitro production of HEV RdRp, ORF4 and human TSG101 proteins. A coupled in vitro transcription-translation kit (TNT T7 kit) was used, following manufactures guidelines (Promega, Wis., USA). Aliquots of the TNT expressed proteins were mixed with 2× Laemelli buffer [125 mM Tris (pH 6.8), 4% SDS, 20% glycerol, 100 mM DTT and 0.02% bromophenol blue], incubated at 950 C for 5 minutes, resolved by SDS PAGE and western blotted with anti-myc antibody to confirm protein expression.

1.3. Chemical Synthesis of tat Conjugated Cyclic Peptide 11 (CP11)

Linear peptide 11 (sequence: CGWIYWNV; SEQ ID NO:1) was chemically synthesized, cyclized and conjugated with the linear Tat peptide (sequence: CGRKKRRQRRRPPQ; SEQ ID NO:2) at LifeTein, following previously reported protocol. The resulting Tat-tagged cyclic peptide 11 (CP11) was purified by HPLC (High performance liquid chromatography), and its identity was confirmed by Mass Spectrometry. Stock solution of CP11 was prepared in water and stored in single use aliquots at −200 C.

1.4. Determination of Inhibitory Effect of CP11 on HEV Budding

1.4.1 In-Vitro his-Pull Down Assay [CP11 is Specific Inhibitor of ORF3-TSG101 Interaction]

An in vitro His-pull down assay was performed to monitor whether cyclic peptide inhibits the interaction between HEV ORF3 and human TSG101 proteins. His tagged ORF3 protein was purified from E. coli BL21 (DE3) following previously described protocols (50). Ni-NTA super flow agarose (Thermo Scientific, Massachusetts, USA) bound ORF3-his was equally aliquoted into separate 1.5 ml tubes followed by addition of TNT generated mock lysate, RdRp-myc lysate, TSG101-myc lysate, ORF4-myc lysate and CP11 (1 μM or 10 μM) in the required combinations in phosphate buffered saline (PBS; 10 mM PO43-, 137 mM NaCl, 2.7 mM KCl) supplemented with protease inhibitor cocktail. Samples were incubated on a flip flop shaker at 4° C. for 1 hour, followed by three washes in PBS. 30p1 of 2× Laemelli buffer was added to the beads and incubated at 950 C for 5 minutes. Aliquots of the eluted proteins were resolved by SDS-PAGE and western blotting was done using anti-myc and anti-ORF3 antibodies.

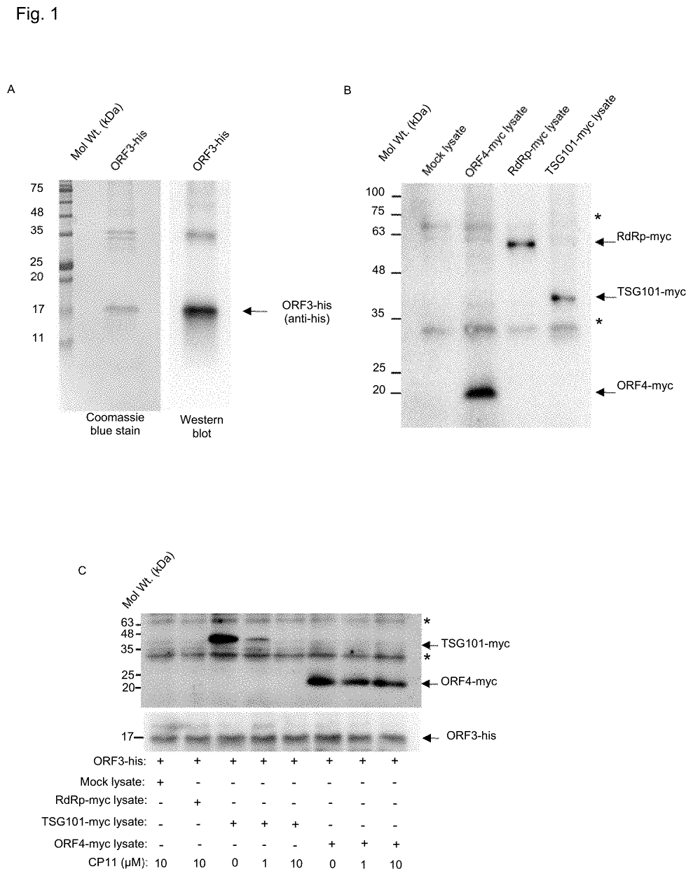

Empty pGBKT7 vector was translated in parallel (denoted as mock lysate) to rule out non-specific cross reactivity of myc antibody with proteins present in the rabbit reticulocyte lysate. RdRp, TSG101 and ORF4 plasmid containing samples produced bands corresponding to their expected size (FIG. 1B). Two non-specific bands were detected in all samples (denoted by *). As reported earlier, ORF3 could pull down both TSG101 and ORF4 (39, 48), indicating its interaction with both the proteins (FIG. 1C, upper panel). Inventors further observed that on addition of 1 μM in the reaction mix, interaction between ORF3 and TSG101 is significantly reduced whereas interaction between ORF4 and ORF3 remains unaffected. On addition of 10 μM of CP11 in the reaction mixture this interaction is completely inhibited confirming specific inhibition by CP11 (FIG. 1C, upper panel). Aliquots of the pull down samples were western blotted using anti-ORF3 antibody to show the amount of ORF3 protein used as bait in different samples (FIG. 1C, lower panel).

1.4.2. Effect of CP11 on HEV Budding

Inventors were aware of the fact that interaction between ORF3 and TSG101 in crucial for the release of g-3 HEV, hence evaluated the effect of CP11 on HEV budding. [Nagashima et al (40, 41)].

The genomic RNAs of p6 HEV and p6 HEV-Luc were synthesized in vitro and size/integrity was monitored by formaldehyde agarose gel electrophoresis. ORF4-Huh7 cell line were prepared as reported (48) and maintained in Dulbecco's modified eagle medium containing 10% fetal calf serum, 50 IU/ml penicillin and streptomycin in 5% CO2. 200 μg/ml hygromycin was added to the cells during routine maintenance. Cells were then electroporated with genomic RNAs of p6 HEV and p6 HEV-Luc in a 4 mm cuvette at 200 Volt, 950 μF and infinite resistance. Electroporated cells were maintained in complete media for 5 days prior to the treatment with CP11.

For infection studies, the HEV infection model was prepared by infecting 4×105 ORF4-Huh7 cells with 8×106 genome copies of the g-1 HEV clinical isolate for one hour, followed by three times washing of infected cells in PBS and maintenance in complete media for 5 days prior to treatment with CP11.

On 6th day post-electroporation/post-infection, HEV infection model was treated every alternate day with 10 μM of CP11 for 6 days followed by measurement of cell viability, quantification of replication of the viral genome and release of the progeny viruses.

For estimation of the amount of virus released to the culture medium, medium from the above samples were collected and clarified by centrifugation at 7197×g, 4° C. for 15 minutes. 8% PEG (polyethylene glycol) 6000 and 0.4 M NaCl (final concentration) was added to the clarified samples and the mixture was incubated at 0° C. for 16 hours, followed by centrifugation at 20,000×g for 30 minutes. The pellet was resuspended in PBS and RNA was isolated using QIAamp Viral RNA Mini kit. Intracellular RNA was isolated using TRI reagent (MRC, Ohio, USA), following manufactures protocol.

For measuring levels of p6 HEV sense RNA and host GAPDH RNA, cDNA was synthesized using random hexamers and Superscript III reverse transcriptase. For subsequent quantitative real time, FP of sequence “atgcgaattccgcgccgttgtaacct” (SEQ ID NO:3) and RP of sequence “accgggacagcgtgga” (SEQ ID NO:4) were used for p6 HEV sense and FP of sequence “gagtcaacggatttggtcgt” (SEQ ID NO:5) and RP of sequence “ttgattttggagggatctcg” (SEQ ID NO:6) were used for host GAPDH RNA.

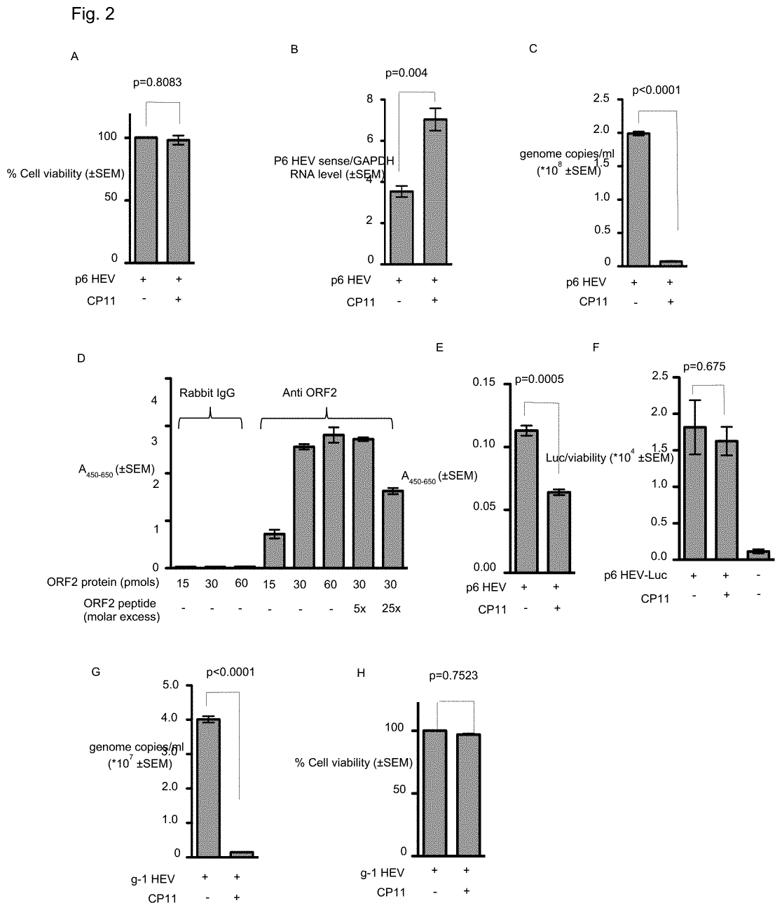

Cell viability was measured by MTT [3-(4,5-dimethyl-2-thiazolyl)-2,5-diphenyl-2H-tetrazolium bromide] assay using cell titer 96 non-radioactive cell proliferation assay kit. MTT assay of p6 HEV expressing Huh7 cells in the presence and absence of CP11 ruled out the possible cytotoxic effect of CP11 (FIG. 2A).

Quantitative real time PCR (QRT-PCR) measurement of viral sense strand RNA level in the same cells revealed its significant increase in the cells treated with CP11 (FIG. 2B). This data indicated that CP11 might be interfering with virus release, which results in intracellular accumulation of the virions or it might be enhancing replication/stability of the virus.

Inventors then designed two different assays to measure the amount of virus released to the culture medium: (a) quantitation of viral genome copies in the viruses present in the culture medium and (b) measurement of the viral capsid protein (ORF2) in the viruses released to the culture medium.

(a) Quantitation of Viral Genome Copies in the Viruses Present in the Culture Medium

Viruses present in the culture medium were precipitated using PEG 6000. Total RNA was isolated from the PEG 6000 precipitated virus and genome copies were estimated. CP11 treatment resulted in approximate 90% reduction in the viral genome copies, indicating its efficiency in inhibiting virus release (FIG. 2C).

A direct readout of the inhibitory effect of CP11 on virus release was obtained by measuring the level of viral ORF2 protein in the PEG 6000 precipitated samples.

(b) Measurement of the Viral Capsid Protein (ORF2) in the Viruses Released to the Culture Medium

An ELISA was standardized using purified recombinant ORF2 protein (112-608aa region) as the antigen and a rabbit polyclonal anti-ORF2 antibody, which was earlier generated and characterized in our laboratory (48). Specificity of the assay was monitored by including appropriate controls such as rabbit preimmune serum and competition with the peptide, which was used to generate the antibody.

For optimization of conditions to assess if our in-house generated ORF2 antibody is suitable for ELISA (enzyme-linked immunosorbent assay) to detect HEV particles, different quantities (15, 30 and 60 pmols) of recombinant ORF2 virus like particles (VLP formed by 112-608 amino acid region of the ORF2 protein) purified from the Pichia pastoris was coated in triplicate onto the wells of an ELISA plate and incubated at 4° C., overnight. Next day, wells were washed twice in PBS, incubated with blocking solution (PBS+1% BSA) at 37° C. for 1 hour, washed three times in wash buffer (PBS+0.1% tween−20), incubated with anti-ORF2 antibody or pre-immune serum in assay buffer (PBS+0.05% tween−20+0.2% BSA) for 1 hour at 37° C., washed four times in wash buffer, incubated with anti-rabbit IgG HRP (horse raddish peroxidase) in assay buffer for 1 hour at 37° C. and washed four times in wash buffer. HRP activity was measured by colorimetry using TMB [3,3′,5,5′-tetramethylbenzidine, (Sigma, Life Science, USA)] as the substrate. Values were measured at A450 using a multimode microplate reader (Synergy HT, BioTek, Vermont, USA). Where indicated, ORF2 peptide was added to the assay buffer during primary antibody incubation step.

ORF2 antibody could specifically detect the antigen, which could be blocked by increasing amount of the antigenic peptide, indicating the suitability of the former for ELISA (FIG. 2D).

For ELISA mediated quantification of p6 HEV present in the culture medium, PEG 6000 precipitated virus was resuspended in PBS and coated onto the ELISA plate overnight at 4° C. Remaining procedure was same as mentioned above. ORF2 level was significantly less in the CP11 treated samples, further supporting the QRT-PCR data (FIG. 2E).

Possible effect of CP11 on HEV replication was investigated using a Huh7 cell-based model of p6 HEV replicon expressing luciferase. Luciferase assay was done through well-known methods. Briefly, Huh7 cells were electroporated with in vitro synthesized capped genomic RNA of p6 HEV-Luc and maintained in complete media for 5 days prior to the treatment with the cyclic peptide. 6th day post electroporation, 10 μM CP11 was added for 48 hours. Gaussia luciferase activity in the culture media was measured using Renilla luciferase assay kit (Promega, Wisconsin, USA). Luciferase values were normalized to that of the cell viability assay, and plotted as mean±SEM. CP11 had no effect on luciferase activity, indicating that it does not affect viral replication/RNA stability (FIG. 2F).

1. A cyclic peptide having the amino acid sequence Cys-Gly-Trp-Ile-Tyr-Trp-Asn-Val (Seq. ID No. 1) for inhibiting interaction between HEV ORF3 and host TSG101.

2. An expression vector coding for the cyclic peptide of claim 1.

3. A pharmaceutical composition for inhibiting HEV infection and proliferation comprising the cyclic peptide of claim 1 in an amount sufficient to inhibit HEV infection and proliferation; and a pharmaceutically-acceptable carrier.

4. A method for treating Hepatitis E in a host, which comprises the step of administering the pharmaceutical composition of claim 3 to the subject in a dose sufficient to inhibit interaction between HEV ORF3 and host TSG101.

5. Use of cyclic peptide of claim 1 for inhibiting infection and proliferation of HEV in a host.

6. Use of cyclic peptide of claim 1 for inhibiting interaction between HEV ORF3 and host TSG101.

7. A pharmaceutical composition for inhibiting HEV infection and proliferation comprising the expression vector of claim 2 in an amount sufficient to inhibit HEV infection and proliferation; and a pharmaceutically-acceptable carrier.

8. A method for treating Hepatitis E in a host, which comprises the step of administering the pharmaceutical composition of claim 7 to the subject in a dose sufficient to inhibit interaction between HEV ORF3 and host TSG101.

9. Use of expression vector of claim 2 for inhibiting infection and proliferation of HEV in a host.

10. Use of pharmaceutical composition of claim 3 for inhibiting infection and proliferation of REV in a host.

11. Use of expression vector of claim 2 for inhibiting interaction between HEV ORF3 and host TSG101.

12. Use of pharmaceutical composition of claim 3 for inhibiting interaction between HEV ORF3 and host TSG101.
